(19) United States
(12) Patent Application Publication
(10) Pub. No.: US 2021/0001306 A1
(43) Pub. Date: Jan. 7, 2021

(54) COMPOSITE MATERIAL FOR BIOSEPARATIONS

(71) Applicant: CHIRAL TECHNOLOGIES EUROPE SAS, Illkirch Cedex (FR)

(72) Inventors: Tong ZHANG, Illkirch Cedex (FR); Pilar FRANCO, Illkirch Cedex (FR); Yasuto MORISHITA, Illkirch Cedex (FR); Klaus GOTTSCHALL, Heddesheim (DE)

(73) Assignee: CHIRAL TECHNOLOGIES EUROPE SAS, Illkirch Cedex (FR)

(21) Appl. No.: 16/978,337

(22) PCT Filed: Mar. 5, 2019
(86) PCT No.: PCT/EP2019/055384

§ 371 Date: Sep. 4, 2020

(30) Foreign Application Priority Data
Mar. 5, 2018 (EP) 18160019.8

(51) Int. Cl. B01J 20/26; C07K 1/16; C07K 16/00; B01J 20/28; B01J 20/285; B01J 20/32; B01D 15/08

(52) U.S. Cl. CPC B01J 20/267; C07K 1/16; C07K 16/00; B01J 20/28083; B01D 15/08; B01J 20/285; B01J 20/3204; B01J 20/3282; B01J 20/28085

(57) ABSTRACT

The present invention relates to composite materials useful for purifying proteins obtained from biological feedstocks. The composite materials of the invention comprise a porous support having an average pore size of 5 to 500 nm, said porous support being filled with a polymer which is cross-linked, wherein the polymer is selected from polyvinylamines or polyallylamines having a weight average molecular weight (Mw) of 2,000 to 500,000 Da and a hydrolysis degree of the formamide groups of at least 66%, with the proviso that a polyvinylamine having a weight average molecular weight (Mw) of 27,200 Da and a hydrolysis degree of 70% and a polyvinylamine having a weight average molecular weight (Mw) of 50,000 Da and a hydrolysis degree of 95% are excluded.

### TECHNICAL FIELD

The present invention relates to composite materials useful for purifying proteins obtained from biological feedstocks.

### BACKGROUND OF THE INVENTION

The relevance of proteins for use as bio-pharmaceuticals has continuously increased during the last decades in many therapeutic and diagnostic applications. One area of particular interest is the use of recombinant monoclonal antibodies (mAbs). The number of approved therapeutic mAbs and fragments thereof for the treatment of inflammatory diseases, diabetes, various cancers and blood disorders increases each year.

Due to the pharmacokinetic properties of mAbs, in many cases initial single doses in the range of about 0.1-1 g per patient are required, followed by a weekly or monthly administration of similar doses. Therefore, large amounts of therapeutic mAbs are needed and thus therapeutic mAbs must be manufactured on an industrial scale. The mAbs are manufactured in biological feedstocks such as fermentation broths (filtrates) and cell cultures which vary in the expression levels of secreted recombinant antibodies and in their impurities content.

To qualify as pharmaceuticals, target proteins must be essentially free of any product- or process-related impurities which are always found in cell culture supernatants or filtrates after harvesting (e.g., cells and cell debris from the secreted target proteins in the culture medium). These contaminants comprise not only proteins and nucleic acids (DNA and RNA) from genetically engineered host, e.g., Chinese Hamster Ovary Host Cell Proteins (CHO-HCPs) and respective DNA (CHO-DNA), but also remaining cell culture supplements, including proteins added as nutrients or stabilizers (e.g., Bovine Serum Albumin—BSA or transferrin), salts, buffers, as well as endotoxins and pathogenic germs or fragments thereof.

The known methods for the purification of target proteins include the removal of viruses, endotoxins and to a certain extent nucleic acids by appropriate membrane filtration steps (e.g., by binding to strong anion exchanger membranes) and the removal of low molecular weight water-soluble contaminants during subsequent unit operations in Downstream Processing (DSP). The complete removal of the broad spectrum of different HCPs is, however, a difficult task, which so far has mainly been solved by the application of multiple dedicated preparative chromatography steps.

The chromatographic purification methods used in the DSP of mAbs and other recombinant protein products include affinity chromatography, cation and anion exchange, hydrophobic interaction, and metal chelate affinity. More recently a variety of multimodal and pseudo-affinity chromatography media became available and found their use in respective production processes, e.g., for product polishing after ion exchange or affinity chromatography steps (EP-A-1807205). In the currently applied chromatographic methods, two classical chromatographic modes are normally found: one based in continuous elution chromatographic processes and the other based on “bind-and-elute” concepts.

The common principle of these chromatographic separation methods is the selective adsorption capabilities of the various chromatography media towards one or more components from the biological samples. Thus, unbound (or weakly bound) components are separated from the (more) strongly bound ones and appear in the corresponding breakthrough fraction. Moreover, bound components can often be separated from each other by adjusting elution conditions to form a continuous or step gradient with increasing or decreasing ionic strength, pH or specific displacer concentration, in order to obtain a volume- and time-based change in conditions leading to selective desorption of individual components.

Among the available chromatographic methods, Size Exclusion Chromatography (SEC) is not considered useful for large scale operations, except for polishing purposes, due to its low productivity, low resolution and low speed. In contrast, one of the most broadly used first steps in the industrial chromatographic mAb purification platforms is based on a “capture” or “bind-and-elute” affinity mechanism. Such process involves the binding of the target compound (“capture”), whereas the majority of the undesired products are left unbound or may be separated from the target by a selective elution step, releasing bound impurities before or after the target substance. A representative example of such bind-and-elute process is the use of Protein A.

In affinity purification of mAbs, immunoglobulins are specifically bound to immobilized Protein A, under conditions favoring very strong binding of the target protein to the chromatographic material, while HCPs and other impurities remain largely unbound. Thus, after washing out the unbound components, the bound immunoglobulins can be released by changing the pH in the respective column from around neutral to rather acidic conditions (e.g., to pH 3) by flushing the column with an appropriate acidic buffer solution. In theory, the collected immunoglobulin product should be entirely pure after this step, due to the extraordinary high and specific selectivity of Protein A for binding to distinct genetically conserved structural motives of the antibody molecules. However, in real technical production processes a number of side effects prevent such perfect one step purification. Among these co-elution of residual HCPs, either bound to Protein A, the chromatography matrix material, or even to immunoglobulins, is observed. Furthermore, leaking of traces of Protein A and degradation products thereof may occur, showing potential to bind to the target proteins again, particularly after the necessary rapid adjustment of the pH back to a range compatible with antibody stability. Exposure of immunoglobulins to the specific process conditions of Protein A chromatography may also favor more or less irreversible product losses, due to intrinsic protein instability, e.g., aggregate formation, partial degradation by proteolysis and other adverse effects.

Thus, additional purification steps are always required, in order to achieve the high purity levels defined for pharmaceutical grade antibody products. These steps, including ion exchange and various multimodal chromatography methods, are necessary to further reduce HCPs and nucleic acid levels, as well as to remove protein aggregates and lower molecular mass antibody degradation products. Such purification steps contribute to further reduction in product yield and add costly operational and time consuming efforts to the overall production process. Thus, there is a need for technologies which can remove the majority of the impurities in a one step process.

A number of methods for the purification of mAbs and other proteins using composite adsorbents are known. In these methods, the composite adsorbents are typically packed into chromatographic columns.

WO95/025574 discloses a method for removing contaminants from a biological fluid comprising bringing said biological fluid into contact with a cross-linked hydrophobic polymeric network overlaying, but not covalently bound to, a porous mineral oxide matrix, having its interior porous volume substantially filled by said hydrophobic network, whereby hydrophobic and amphiphilic molecules with an average molecular mass below 10,000 Da are removed.

U.S. Pat. No. 6,783,962 B1 relates to a particulate material useful for the isolation/purification of bio-macromolecules. The particulate material has a density of at least 2.5 g/ml, the particles of the particulate material have an average diameter of 5-75 μm, and the particles of the particulate material are essentially constructed of a polymeric base matrix and a non-porous core material, said core material having a density of at least 3.0 g/ml. The polymeric base matrix includes pendant groups which are positively charged at pH 4.0 or which are affinity ligands for a bio-molecule.

WO2004/073843 discloses a composite material that comprises a support member that has a plurality of pores and a macroporous cross-linked gel filling the pores of the support member. Also disclosed is a process for adsorbing a biological molecule or a biological ion from a liquid, which comprises passing a liquid containing the biological molecule or biological ion through a composite material which bears binding sites that display specific interactions for the biomolecule on the macroporous gel.

EP-A-2545989 discloses a composite material for chromatographic applications which comprises a porous support and a cross-linked polymer on the surface of the porous support, wherein the ratio between the pore size [nm] of the porous support and the cross-linking degree [%] of the cross-linked polymer is from 0.25 to 20 [nm/%], and wherein the cross-linking degree is from 5 to 20% based on the total number of cross-linkable groups in the cross-linked polymer.

WO 2018/050849 discloses the preparation of a composite material comprising porous silica gel with a pore size of 25 nm and a cross-linked poly(vinylformamide-co-polyvinylamine) with an average molecular weight of 27,200 Da and a hydrolysis degree of 70% (Example 1). In the general part of the document, a polyvinylamine with an average molecular weight of 50,000 Da which is hydrolyzed to 95% is also mentioned.

US-A-2017/304803 discloses a sorbent comprising a porous support material coated with an amino group-containing polymer such as polyvinylamine. However, this reference does not mention polyvinylamines with a hydrolysis degree of the formamide groups of at least 66%.

Dragan, E. S. et al., Macromol. Rapid Commun., 2010, vol. 31, pp. 317-322 describes the production of a composite material comprising silica microparticles with an average particle size of 15 to 40 μm and a maximum pore diameter in the range of 4 to 6 nm, which are coated with a cross-linked polyvinylamine. This reference teaches that the inner pores of the silica are inaccessible to the polymer chains.

EP-A-2027921 describes a porous sorptive media comprising a substrate having a first external side and a second external side, both sides being porous, and a porous thickness between them, said substrate having a sorptive material substantially covering the solid matrix of the substrate and said first and second external surfaces, said sorptive material comprising a crosslinked polymer having attached primary amine groups. Particulate material substrates are not mentioned in this reference.

The present invention has been designed to overcome the limitations of existing technologies in the purification of bio-molecules.

### SUMMARY OF THE INVENTION

The object of the present invention is to provide composite materials which achieve improved purification of proteins such as mAbs from biological feedstocks containing same.

The object of the present invention is achieved by a composite material according to appended claim 1.

Specifically, the present invention provides a composite material comprising:
a porous support having an average pore size of 5 to 500 nm, said porous support being filled with a polymer which is cross-linked,
wherein the polymer is selected from polyvinylamines or polyallylamines having a weight average molecular weight (Mw) of 2,000 to 500,000 Da and a hydrolysis degree of the formamide groups of at least 66%,
with the proviso that a polyvinylamine having a weight average molecular weight (Mw) of 27,200 and a hydrolysis degree of 70% and a polyvinylamine having a weight average molecular weight (Mw) of 50,000 and a hydrolysis degree of the formamide groups of 95% are excluded.

It was surprisingly found that by combining the specified porous support with the specified cross-linked polymer, a composite material with enhanced purification capability can be obtained.

The present invention provides a composite material for purification of target proteins from undesired compounds contained in the same solution or suspension. The composites are particularly suited for the efficient removal of impurities from manufactured biotherapeutics, such as mAbs, and could easily be integrated in clarification or downstream purification processes (DSP).

The composite materials can preferably simultaneously deplete DNA and HCPs from the protein-containing solutions obtained during protein production and can also achieve excellent protein recovery.

The invention is also directed to a method for producing the composite material comprising the steps of:

a) soaking a porous support having an average pore size of 5 to 500 nm with a solution or a dispersion containing a polymer, a cross-linker, and a solvent; and

b) cross-linking the polymer with the cross-linker at a temperature below 250° C.,
wherein the polymer is selected from polyvinylamines or polyallylamines having a weight average molecular weight (Mw) of 2,000 to 500,000 Da and a hydrolysis degree of the formamide groups of at least 66%,
with the proviso that a polyvinylamine having a weight average molecular weight (Mw) of 27,200 Da and a hydrolysis degree of the formamide groups of 70% and a polyvinylamine having a weight average molecular weight (Mw) of 50,000 Da and a hydrolysis degree of the formamide groups of 95% are excluded.

Also provided is the use of the composite material of the invention for purifying a target protein in a feedstock.

Further, the invention provides a method for purifying a target protein in a feedstock, said method comprising the steps of:

i) contacting the feedstock with a composite material of the invention for a sufficient time;

ii) separating the composite material from the purified feedstock;

iii) optionally, isolating the purified target protein from the feedstock; and

iv) optionally, washing the composite material with a solvent and collecting the obtained solution for further processing.

### DETAILED DESCRIPTION

Composite Material

In the present specification, the terms “composite”, “composite material” and “adsorbent” are used interchangeably.

In the present specification, any reference to a “pore size” means “average pore size”.

The porous support material has an average pore size of 5 nm to 500 nm. In combination with any of the above or below embodiments, the average pore size is preferably 15 nm to 300 nm, more preferably 20 nm to 200 nm, further preferably 25 nm to 250 nm, even more preferably 30 nm to 200 nm, and most preferably 40 rim to 100 nm. In the present specification, the average pore size of the porous support material is determined by mercury intrusion according to DIN 66133.

The porous support material can be a membrane, a hollow-fiber, a non-woven tissue, a monolithic or a particulate material. Particulate and monolithic porous materials are preferred. In a preferred embodiment in combination with any of the above or below embodiments, the porous support material is a particulate porous support material which has irregular or spherical shape.

In a further preferred embodiment, in combination with any of the above or below embodiments, the porous support material is composed of a metal oxide, a semi-metal oxide, a ceramic material, a zeolite, or a natural or synthetic polymeric material.

In a further preferred embodiment, in combination with any of the above or below embodiments, the porous support material is porous silica, alumina or titania particles.

In a further preferred embodiment, in combination with any of the above or below embodiments, the porous support material is porous silica gel.

In a further preferred embodiment, in combination with any of the above or below embodiments, the porous support material is a porous polysaccharide, such as cellulose, chitosan or agarose.

In a further preferred embodiment, in combination with any of the above or below embodiments, the porous support material is a porous synthetic polymer, such as polyacrylate, polymethacrylate, polyetherketone, polyalkymether, polyarylether, polyvinylalcohol, or polystyrene, or mixtures or copolymers thereof.

In a further preferred embodiment, in combination with any of the above or below embodiments, the porous support material is a particulate material with an average particle size (diameter) of 1 μm and 500 μm, preferably between 20 μm and 200 μm, more preferably 30 to 150 μm and most preferably 35 to 100 μm.

In the present specification, the average particle size (diameter) and the particle size distribution of the porous support is determined by Malvern Laser Diffraction.

In the present specification, unless otherwise specified, the term “polymer” refers to the polymer before being cross-linked.

In the present specification, the term “hydrolysis degree” refers to the “hydrolysis degree of the formamide groups of the polymer.

The composite material of the present invention comprises a polymer which is cross-linked. Said polymer (before being cross-linked) is selected from polyvinylamines or polyallylamines having a weight average molecular weight (Mw) of 2,000 to 500,000 Da and a hydrolysis degree of the formamide groups of at least 66%.

In the present specification, the polyvinylamines and polyallylamines include linear or branched homopolymers of vinylamine or allylamine and copolymers of vinylamine or allylamine and an amino- or amido-groups.

In a further preferred embodiment, in combination with any of the above or below embodiments, the polyvinylamine is a linear or branched homopolymer of vinylamine or a copolymer of vinylamine and vinylformamide. Preferably, the copolymer of vinylamine and vinylformamide comprises 1% to 70% vinylformamide units, more preferably 2% to 40% vinylformamide units, most preferably 5% to 25% vinylformamide units, based on the total number of structural units of the polymer. In a further preferred embodiment, in combination with any of the above or below embodiments, the polyallyamine is a linear or branched homopolymer of allylamine or a copolymer of allylamine and allylformamide. Preferably, the copolymer of allylamine and allylformamide comprises 1% to 70% allylformamide units, more preferably 2% to 40% allylformamide units, most preferably 5% to 25% allylformamide units, based on the total number of structural units of the polymer.

In a preferred embodiment, in combination with any of the above or below embodiments, the weight average molecular weight (Mw) of the polyvinylamine or polyallylamine is 2,000 to 500,000 Da, preferably 15,000 to 400,000 Da, more preferably 20,000 to 300,000 Da, most preferably 25,000 to 250,000 Da.

In the present specification, the weight average molecular weight (Mw) of a polymer is determined by size exclusion chromatography (SEC) coupled to multi-angle-light scattering and refractive index detectors (SEC-MALS-RI).

In a preferred embodiment, in combination with any of the above or below embodiments, the hydrolysis degree of the formamide groups of the polyvinylamine or polyallylamine is 67% to 99%, more preferably 68% to 94%, even more preferably 72% to 90%, and most preferably 75% to 86%.

In the present specification, the hydrolysis degree of the formamide groups of the polymer is determined by ¹H-NMR according to the following method:

5.25 g of the polymer is weighted into a flask and 10 ml of water is added. The obtained mixture is rotated to get a homogenous composition and finally evaporated at 50° C. under vacuum until a dry solid is observed. The solid is dried under high vacuum (0.1 mbar) in an oven at 80° C. for 15 h to yield a dry residue.

The degree of hydrolysis is determined by ¹H-NMR (400 MHz apparatus from Brucker, solvent: D₂O) based on the quantification of hydrolysed groups versus total hydrolysable groups according to the method described in reference:
Q. Wen, A. M. Vincelli, R. Pelton, “Cationic polyvinylamine binding to anionic microgels yields kinetically controlled structures”, J Colloid Interface Sci. 369 (2012) 223-230.

In a further preferred embodiment, in combination with any of the above or below embodiments, the weight average molecular weight (Mw) of the polyvinylamine or polyallylamine is 15,000 to 80,000 Da, preferably 20,000 to 70,000 Da, more preferably 25,000 to 50,000 Da and the hydrolysis degree of the formamide groups is 66% to 90%, preferably 67% to 80%, more preferably 68% to 75%.

In a further preferred embodiment, in combination with any of the above or below embodiments, the weight average molecular weight (Mw) of the polyvinylamine or polyallylamine is 100,000 to 500,000 Da, preferably 150,000 to 400,000 Da, more preferably 200,000 to 300,000 Da and the hydrolysis degree of the formamide groups is 70% to 99%, preferably 75% to 95%, more preferably 75% to 90%.

In a further preferred embodiment, in combination with any of the above or below embodiments, the first polymer is cross-linked to a cross-linking degree of 5 to 25% (mol/mol). In a preferred embodiment, in combination with any of the above or below embodiments, the cross-linking degree is 6 to 15% (mol/mol), preferably 7 to 12% (mol/mol), more preferably 8 to 9% (mol/mol).

In the present specification, the “cross-linking degree” is defined as the cross-linker/polymer ratio (also referred to as “cross-linker ratio”). The “cross-linker ratio” is defined as the percentage in mol of the cross-linker versus the vinylamine structural units present in the polymer solution (based on average molecular weight) used for the reaction.

That is, the cross-linker ratio is calculated by the following formula (1):

$$cross linker ratio = \frac{V 1 \times d 1 \times C 1 \times Mw 2}{W 2 \times C 2 \times Mw 1} \times 100 \% \qquad ( 1 )$$

wherein V1 (ml) is the volume of cross-linker, d1 (g/ml) is the density of cross-linker, C1 (wt %) is the concentration of cross-linker, W2 (g) is the weight of polymer solution, C2 (wt %) is the concentration of polymer, Mw1 (g/mol) is the molecular weight of cross-linker and Mw2 (g/mol) is the average monomer unit molecular weight.

Mw2 is calculated by the following formula (2):

Mw2=(ΣₖNk×Mk)/ΣₖNk (2)

wherein Nk is the number of monomer units of type k forming the polymer and Mk is the molecular weight (g/mol) of a monomer unit of type k.

The cross-linked polymer may be derivatized with functional groups other than amino- or amido-groups. However, the cross-linked polymer is preferably not derivatized with such functional groups.

In a preferred embodiment, in combination with any of the above or below embodiments, the concentration of cross-linked polymer is at least 3% w/w, preferably at least 5% w/w, more preferably, at least 7% w/w, and is preferably less than 25% w/w, more preferably less than 20% w/w, most preferably less than 15%, based on the total weight of the dry composite material.

Method for Producing the Composite Material

The composite material of the present invention can be produced according to the following method:

a) soaking a porous support having an average pore size of 5 to 500 nm with a solution or a dispersion containing a polymer, a cross-linker, and a solvent; and

b) cross-linking the polymer with the cross-linker at a temperature below 250° C.,
wherein the polymer is selected from polyvinylamines or polyallylamines having a weight average molecular weight (Mw) of 2,000 to 500,000 Da and a hydrolysis degree of the formamide groups of at least 66%, with the proviso that a polyvinylamine having a weight average molecular weight (Mw) of 27,200 Da and a hydrolysis degree of the formamide groups of 70% and a polyvinylamine having a weight average molecular weight (Mw) of 50,000 Da and a hydrolysis degree of the formamide groups of 95% are excluded.

Any cross-linker having at least two reactive groups can be used in the present invention.

In a preferred embodiment, in combination with any of the above or below embodiments, the cross-linker is selected from bis-epoxides, dialdehydes, and diglycidylethers. In a more preferred embodiment, in combination with any of the above or below embodiments, the cross-linker is selected from propanediol diglycidylether, butanediol diglycidylether, hexanediol diglycidylether, polyethylene glycol diglycidyl ether, glutaric dialdehyde and succinic dialdehyde. More preferably, the cross-linker is selected from butanediol diglycidylether and hexanediol diglycidylether.

In a preferred embodiment, in combination with any of the above or below embodiments, the cross-linker ratio is 6 to 15% (mol/mol), more preferably 7 to 12% (mol/mol), and most preferably 8 to 9% (mol/mol).

Any solvent or medium capable of dissolving or dispersing the polymer and the cross-linker may be used provided that it does not react or only slowly reacts with the cross-linker and the polymer under the conditions of step b) of the above method. Slowly, in this context, means that no observable reaction between the cross-linker and the solvent and between the polymer and the solvent occurs for the duration of step (b).

In a preferred embodiment, in combination with any of the above or below embodiments, the solvent is a polar protic or a polar aprotic solvent. In a preferred embodiment, in combination with any of the above or below embodiments, the solvent is a polar protic solvent selected from water, Cₜ₋₆ alcohols (e.g. methanol, ethanol, isopropanol, and butanol) and mixtures thereof. Water is most preferred.

In a preferred embodiment, in combination with any of the above or below embodiments, the pH of the polymer-cross-linker solution employed in step a) is adjusted to 8 to 13, preferably 9 to 11, most preferably 10 to 11. The pH adjustment can be carried out by adding a strong base such as NaOH or KOH.

During step b) of the above method, the temperature is preferably between 20 to 180° C., more preferably 40 to 100° C., and most preferably 50° C. and 80° C.

In a preferred embodiment, in combination with any of the above or below embodiments, the duration of step b) is preferably between 1 hour and 100 hours, more preferably between 8 to 60 hours, and most preferably between 18 hours and 48 hours.

In a further preferred embodiment, in combination with any of the above or below embodiments, step b) is carried out at 40 to 100° C. for 8 to 60 hours, preferably at 50 to 80° C. for 12 to 50 hours, more preferably at 60° C. for 24 to 48 hours.

In a further preferred embodiment, in combination with any of the above or below embodiments, the method further comprises a step c) of hydrolysing any unreacted cross-linkable groups of the cross-linker after step b).

Uses of the Composite Material

In the present specification, the terms “feedstock” and “feed” are used interchangeably.

In the present specification, term “proteins” includes polypeptides. Such polypeptides preferably contain at least 20 amino acid residues, more preferably between 40 and 80 amino acid residues.

The composite material of the present invention is useful for purifying a target protein in a feedstock.

In a preferred embodiment, in combination with any of the above or below embodiments, the feedstock comprises host cell proteins (HCPs), and DNA, and optionally RNA and other nucleic acids.

In the present invention, the feedstock optionally contains albumins, endotoxins, detergents and microorganisms, or fragments thereof.

The invention also provides a method for purifying a target protein in a feedstock, said method comprising the steps of:
i) contacting the feedstock with a composite material according to the invention for a sufficient time;
ii) separating the composite material from the purified feedstock;
iii) optionally, isolating the target protein from the feedstock; and
iv) optionally, washing the composite material with a solvent and collecting the obtained solution for further processing.

In a preferred embodiment, in combination with any of the above or below embodiments, the target protein is a recombinant protein such as a monoclonal antibody (mAb) (e.g. Human immunoglobulin (hIgG)).

In a preferred embodiment, in combination with any of the above or below embodiments, the solvent of the feedstock is water optionally containing buffer(s), salt(s) and/or modifier(s).

In a preferred embodiment, in combination with any of the above or below embodiments, the feedstock is a fermentation broth supernatant (before or after filtration) or a cell culture supernatant (CCS) comprising the target protein and DNA, RNA, or other nucleic acids, and Host cell proteins (HCPs) as impurities.

In a preferred embodiment, in combination with any of the above or below embodiments, the composite material is used in a batch adsorption process. In this embodiment, in step i) of the purification method of the invention, the composite material is dispersed in the feedstock and in step ii), the composite material is separated from the feedstock (e.g. by centrifugation).

In another preferred embodiment, in combination with any of the above or below embodiments, the composite material is packed in a chromatography column.

In the method for purifying a target protein of the present invention, the feedstock is contacted with the composite material according to the invention for a sufficient time. In a preferred embodiment, in combination with any of the below embodiments, the contact time is 1 min to 10 hours, preferably 3 min to 5 hours, more preferably 5 min to 1 hour.

In a preferred embodiment, in combination with any of the above or below embodiments, prior to contacting the composite material with the feedstock, the composite material is equilibrated in an aqueous solution with a pH below 8, preferably 3 to 7.5, more preferably 4 to 7, and most preferably 5 to 6. The pH of the aqueous solution can be adjusted with any suitable buffer. For example, monobasic acids or salts thereof can be used for adjusting the pH. Preferred monobasic acids are formic, acetic, sulfamic, hydrochloric, perchloric acid, and glycine. Preferred salts of the monobasic acids are ammonium, alkyl ammonium, sodium and potassium salts.

In a preferred embodiment, in combination with any of the above or below embodiments, the pH is adjusted with ammonium acetate.

In a further preferred embodiment, in combination with any of the above or below embodiments, the pH is adjusted with phosphate-buffered saline (PBS).

In a preferred embodiment, in combination with any of the above or below embodiments, the ratio of feedstock to composite material (volume of feed to weight of dry composite material) is in the range of 2:1 to 100:1, preferably 5:1 to 80:1, more preferably 10:1 to 70:1, most preferably 20:1 to 50:1. High ratios of feedstock to composite material are preferred from the viewpoint of achieving efficient utilization of the composite material.

In a preferred embodiment, in combination with any of the above or below embodiments, the composite material separated in step ii) of the above method, which contains adsorbed impurities, is subjected to an elution procedure to elute said impurities, thereby regenerating the composite material for further use.

The method for purifying a target protein of the present invention may contain additional purification steps known in the art. Examples of such purification steps include ion exchange chromatography, addition of flocculation or precipitation agents, centrifugation, crystallization, affinity chromatography (e.g. employing separation media harboring Protein A, Protein G, or a combination thereof), membrane filtration, depth filtration (with diatomaceous earth or activated carbon) and application of a monolithic separation agent.

In a preferred embodiment, in combination with any of the above or below embodiments, steps i) and ii) of the method for separating a target protein of the invention are repeated in sequence multiple times (e.g. 2, 3, 4, 5, 6 times) using the same or different composite materials according to the present invention.

The following examples illustrate the invention.

### EXAMPLES

Starting Materials Used in the Examples

The following starting materials have been used in the preparation of the composite materials of the examples:

Polymer:

A1 Lupamin 4570 (supplied by BASF) (a co-polymer of vinylamine and vinylformamide)

A2 Lupamin 4570 further hydrolyzed to 68% hydrolysis degree

A3 Lupamin 4570 further hydrolyzed to 86% hydrolysis degree

A4 Lupamin 4570 further hydrolyzed to 99% hydrolysis degree

Polymers A2 to A4 were obtained by further hydrolysing polymer A1 as follows.

Polymer A1 was homogenized by gentle agitation for 30 min on a rotation station. A weighed amount of the homogenised polymer was placed in a round flask and a sodium hydrate solution in water was added and heated at 80° C. for several hours under the protection of N₂ stream. The mixture was subsequently cooled at room temperature (23° C.) and the pH adjusted by using a hydrochloric acid solution. The exact conditions are listed in Table 1.

TABLE 1
Experimental conditions for obtaining polymers A2 to A4

| | Start- | | | | | Conc. | |
|---|---|---|---|---|---|---|---|
| | ing | Hydro- | Amount | Sodium | Conditions | HCl | Fi- |
| Pol- | pol- | lysis | (g) in | hydrate | (Temp/ | solu- | nal |
| ymer | ymer | degree | reaction | solution | time) | tion | pH |
| A2 | A1 | 68% | 1150 | 300 g/10% | 80° C./5 h | 10% | 9.5 |
| A3 | A1 | 86% | 500 | 100 g/30% | 90° C./72 h | 25% | 9.5 |
| A4 | A1 | 99% | 500 | 100 g/48% | 90° C./24 h | 25% | 11-12 |

The properties of the polymers A1 to A4 are given in Table 2 below.

TABLE 2
Properties of polymers A1 to A4

| | Polymer | | | | |
|---|---|---|---|---|---|
| | hydrolysis | Polymer | dn/dc | | |
| Polymer | degree*¹⁾ | concentration*²⁾ | (ml/g) | Mw | Mw/Mn |
| A1 | 65% | 15% | 0.2997 | 29,600 | 1.8 |
| A2 | 68% | 11% | 0.3104 | 25,200 | 1.2 |
| A3 | 86% | 10% | 0.4219 | 25,200 | 1.3 |
| A4 | 99% | 7% | 0.4219 | 25,000 | 1.2 |

*¹⁾The degree of hydrolysis was determined by ¹H-NMR.
*²⁾The concentration was estimated based on the elemental analysis results.

1) Hydrolysis Degree

The hydrolysis degree of the formamide groups of polymers A1 to A4 was determined by ¹H-NMR as follows.

The polymer samples were prepared for NMR analysis with the following general protocol:

5.25 g of the commercial or further hydrolysed polymer was weighted into a flask and 10 ml of water were added. The mixture was rotated to get a homogenous composition and finally evaporated at 50° C. under vacuum until the dry solid was observed. The solid was dried under high vacuum (≤0.1 mbar) in an oven at 80° C. for 15 h to yield a dry residue.

The degree of hydrolysis was determined by ¹H-NMR based on the quantification of free amine groups versus formamide groups according to the method described in reference:
Q. Wen, A. M. Vincelli, R. Pelton, “Cationic polyvinylamine binding to anionic microgels yields kinetically controlled structures”, J Colloid Interface Sci. 369 (2012) 223-230.

The ¹H-NMR system used for the measurements was a 400 MHz. The dry sample was dissolved in D₂O.

2) Polymer Concentration

The polymer concentration of polymers A1 to A4 was determined based on elemental analysis. The samples were prepared with the same protocol described in the ¹H-NMR section until getting a dry residue. The elemental analyser was a FLASH 2000 Organic Elemental Analyzer (Thermo Scientific).

3) Weight-Average Molecular Weight (Mw), Polydispersity (Mw/Mn), and Specific Increment of Refractive Index (dn/dc) of the Polymers

The weight-average molecular weight (Mw), polydispersity (Mw/Mn), and specific increment of refractive index (dn/dc) of the polymers are determined as follows.

Size exclusion chromatography (SEC) coupled to multi-angle-light scattering and refractive index detectors (SEC-MALS-RI) was used to determine the weight-average molecular weight (Mw) using the Rayleigh-Gans-Debye equation with Zimm formalism.

In this approach the light scattering signal is assumed to be proportional to average molecular weight and sample concentration at any point in a chromatogram, and specific increment of refractive index (dn/dc). Thus, light scattering detectors coupled with a refractive index detector as a concentration detector can accurately determine the average molecular weight for any point in the chromatogram and analysis of the entire chromatographic distribution can be used to determine the weight-average molecular weight (Mw) when the value of dn/dc is obtained.

In the Rayleigh-Gans-Debye equation (Equation (1)), the light scattering signal in proportional to average molecular weight and sample concentration at any point in the chromatogram and specific increment of refractive index (dn/dc).

R(θ)=K*MCP(θ)[1−2A₂MCP(θ)] (1)

In Equation (1), R(θ) is the excess (from the solute alone) Rayleigh ratio (i.e. the ratio of the scatter and incident light intensity, corrected for size of scattering volume and distance from scattering volume), M is molar mass (molecular weight), C is analyte concentration, K* is the Rayleigh ratio constant, determined according to Equation (2)

K*=(4π²(nₒ)²/N_{A}(λ₀)⁴)(dn/dc) (2)

In Equation (2), nₒ is the solvent refractive index, N_{A} is the Avogadro number, λ₀ is the vacuum wavelength of incident light, dn/dc is the specific refractive index increment, P(θ) is the form factor or scattering function and relates the angular variation in scattering intensity to the mean square radius (r_{g}) of the particle, A₂ is a second viral coefficient, a measure of solute solvent interaction.

From this analysis, number average molecular weight (Mn), weight-average molecular weight (Mw), polydispersity (Mw/Mn), and peak molecular weight (Mp) can be determined.

Instrumentation:

SEC/MALS/RI system was composed of Shimadzu LC 20A system, Wyatt Optilab rEX RI detector and Wyatt DAWN HELEOS-II MALS detector.

Molecular weight (Mw and Mn) and polydispersity (Mw/Mn) were calculated using Astra (Version: 5.3.4.20) software from Wyatt.

Tosoh TSKgeI G3000PW×L (7 μm, 7.8 mm I.D×30 cm) with pre-column Tosoh TSKgeI G6000PW×L (13 μm, 7.8 mm I.D×30 cm) were used for SEC analysis of the polymers.

Analytical Conditions:

Mobile phase: 0.45 M sodium nitrate aq.+0.5% (v/v) trifluoroacetic acid (TFA),

Flow rate: 0.5 mL/min

Detection:

Wavelength of linearly polarized laser in MALS: 658 nm

RI

Temperature: 25° C.

Injection volume: 50 μL

Sample dilution: 10 mg of the polymer (at concentration as indicated in Table 2) diluted by 1.5 mL mobile phase

Run time: 58 min

Porous Support:

B1 Silica Gel Davisil LC 250, 40-63 μm (supplied by W. R. Grace)

B2 Silica Gel XWP500A, 35-75 μm (supplied by W. R. Grace)

B3 Silica Gel XWP1000A, 35-75 μm (supplied by W. R. Grace)

The properties of the porous supports B1 to B3 are given in Table 3 below.

TABLE 3
Properties of the porous supports

| Porous | | Particle size |
|---|---|---|
| support | Pore size | distribution |
| B1 | 250 Å | 40-63 μm |
| B2 | 500 Å | 35-75 μm |
| B3 | 1,000 Å | 35-75 μm |

The pore size of the porous support was determined by mercury intrusion according to DIN 66133.

The particle size distribution of the porous support was determined by Malvern Laser Diffraction.

Cross-Linker:

1,6-hexanediol diglycidylether (HDGE; ipox RD18 supplied by Ipox Chemicals)

1,4-butanediol diglycidylether (BDGE; supplied by Sigma-Aldrich and ipox RD3 supplied by Ipox Chemicals)

### Example 1

15 ml of aqueous solution of polymer A2 (11% of polymer A2 in the solution) was mixed with a solution (704 μl) of 1,6-hexanediol diglycidylether (HOGE) to reach 7-9% of the cross-linker. The cross-linker ratio was calculated considering the amount of reacting groups versus the vinylamine units present in the polymer solution used for the reaction. After mixing, the pH was adjusted to 11 with 0.5 M NaOH.

10 g of the dry powder porous support B1 were sedimented in a flat bottom stainless steel dish with 8 cm diameter. The porous support B1 was impregnated with 39.5 g of the polymer-cross-linker solution which was added dropwise and equally distributed over the porous support and mixed using a spatula. The resultant paste was shaken for 1 min on a gyratory shaker at 600 rpm, in order to obtain a homogeneous mass with smooth surface. After covering the dish with a stainless steel lid, the paste was heated without further mixing or moving for 48 hours in an oven at 60° C. yielding 49.6 g of moist composite.

Subsequently 41.3 g of this moist composite was washed on a frit with five times 25 ml of water. Then, the composite cake was suspended in 31.6 ml of 10% sulphuric acid and treated in a shaker bath for two hours at ambient temperature (23° C.) in order to hydrolyse unreacted epoxy groups. Finally, the product was washed on a frit with once more five times 25 ml of water and then stored in 20% ethanol-water.

### Examples 2 to 4 and Comparative Examples 1 and 2

Examples 2 to 4 and Comparative Examples 1 and 2 were prepared in the same way as Example 1 but using the starting materials listed in Table 4.

TABLE 4

| | | Porous | Cross-linker | |
|---|---|---|---|---|
| | Polymer | support | ratio | Cross-linker |
| Example 1 | A2 | B1 | 7-9% | HDGE |
| Example 2 | A3 | B1 | 7-9% | HDGE |
| Example 3 | A2 | B2 | 7-9% | BDGE |
| Example 4 | A2 | B3 | 7-9% | BDGE |
| Comp. Example 1 | A1 | B1 | 7-9% | HDGE |
| Comp. Example 2 | A1 | B1 | 7-9% | BDGE |

Determination of the Depletion Performance and hIgG Recovery of the Composite Materials of the Examples

In order to measure the purification capability of the composite materials, the degree of depletion (separation) of impurities or undesired compounds from the target substance is determined. For this purpose the concentration of individual components in the feed is determined using selective assays. After the purification step, this concentration measurement is repeated with the purified fraction. Thus, it is possible to calculate both purity and recovery from these concentrations and the related volumes.

Feed

The feed was an untreated and undiluted Cell Culture Supernatant CHO-K1 spiked at 2 mg/ml of hIgG from human blood plasma (Octagam, 10% solution, Octapharma, Vienna).

Cell Culture Supernatants (CCSs)

CCS1

CHO-K1, Invivo, Berlin

Cell Line CHO-K1 (2.5×10⁶ viable cells/mi)

Conductivity: 15 mS/cm

Average Host Cell Protein (HCP) concentration 100-150 μg/ml

Average DNA concentration between 700-1,000 ng/ml

CCS2

CHO-K1, Invivo, Berlin

Cell Line CHO-K1

Conductivity: 13 mS/cm

Average Host Cell Protein (HCP) concentration 65-82 μg/mI

Average DNA concentration between 250-500 ng/ml

All adsorbents were equilibrated with 50 mM ammonium acetate at pH 6.5 prior to contacting with the feed.

200 mg of the adsorbent were incubated with 1 ml of the feed using an Eppendorf or a centrifugation tube. The ratio of feed volume and adsorbent weight was 5:1 (1 ml feed:0.2 g adsorbent). After 5 min of gentle shaking, the supernatant was separated by centrifugation for subsequent analysis. A higher ratio of feed volume and adsorbent weight of 50:1 (1 ml feed:0.02 g adsorbent) was also tested. Unless otherwise specified, contact time is 5 min.

To determine the efficiency of depletion of host cell proteins (HCPs) and DNA, as well as the hIgG recovery, the quantification of the above three substances was performed in the raw feed and the depleted feed, after a specified contact time with the composite material. Both values were subsequently compared.

Host Cell Protein (HCP) Determination

Cygnus CHO HCP Elisa Kit 3G was used to determine the efficiency of depletion of host cell proteins (HCPs), CHO Host Cell Proteins 3^{rd} Generation (#F550), from Cygnus Technologies, Southport (USA) according to the manufacturer's instructions (manual “800-F550, Rev. 3, 21 Jul. 2015”), on a VictorX Spectrophotometer and corresponding software from PerkinElmer (Courtaboeuf, France) for reading and data evaluation. Samples were diluted in the sample diluent (Product catalog number #1028 purchased from Cygnus Technologies).

The HCP depletion is expressed as:

HCP depletion (%)=100×(HCP concentration in supernatant)/(HCP concentration in initial spiked CCS)

In the above formula, “supernatant” refers to the purified CCS.

DNA Determination

The samples to be analysed were the starting CCSs (with or without hIgG spiked) and the depleted samples.

The DNA quantification was accomplished utilizing DNA-specific fluorescence assay using Quant-iT™ PicoGreen® dsDNA Reagent Kit (#P7589), Invitrogen (Germany) after DNA extraction with the DNA Extraction Kit (#D100T), Cygnus Technologies, Southport (USA), according to the manufacturer's instructions, on a VictorX Spectrophotometer and corresponding software from PerkinElmer (Courtaboeuf, France) for reading and data evaluation.

The DNA depletion is expressed as:

DNA depletion (%)=100×(DNA concentration in supernatant)/(DNA concentration in initial spiked CCS)

In the above formula, “supernatant” refers to the purified CCS. hIgG recovery determination by Size Exclusion Chromatography (SEC) The recovery of hIgG was determined by quantitative SEC as follows.

The concentration of hIgG in the feed and the recovery rate of hIgG in the purified solution have been determined with SEC under the following conditions.

Column: TSKgel UP-SW3000 4.6×300 mm (particle size 2 μm) from Tosoh Bioscience LCC.

Mobile Phase: 100 mM sodium phosphate pH 6.7 buffer+100 mM Na₂SO₄+0.05% NaN₃

Injection volume: 10 μL—sample diluted with the mobile phase.

Flow rate: 0.35 ml/min.

Detector: DAD 280 nm.

Temperature: 25° C.

This column with high efficiency and the associated analytical conditions allow appropriate quantification of the monomer and dimer peaks.

The hIgG recovery is expressed as:

Recovery (%)=100×(hIgG concentration in supernatant)/(hIgG concentration in initial spiked CCS)

In the above formula, “supernatant” refers to the purified CCS.

The results are shown in Table 5.

TABLE 5
Depletion performance and hIgG recovery of the composites
of the examples at 50:1 feed:composite ratio

| | HCP | DNA | Recovery |
|---|---|---|---|
| | depletion (%) | depletion (%) | hIgG (%) |
| Example 1 | 63 | 89 | 96 |
| Comp. Example 1 | 58 | 83 | 96 |

As seen in Table 5, the recovery rates of hIgG at 50:1 feed:composite ratio were nearly quantitative (≥96%).

At a 5:1 feed composite ratio, the composites of Examples 1 to 4 depleted DNA and HCPs from the feedstocks at >95%.

As can be seen in Table 5, Comparative Example 1, which is obtained using polymer A1 (hydrolysis degree of 65%) has HCP and DNA depletion capability which is inferior to the one of Example 1.

Thus, the composite material of the present invention achieves excellent DNA and HCP depletion and hIgG recovery at high feed to composite ratios, and is therefore suitable for the efficient and cost effective purification of target proteins.

1. A composite material comprising:
a porous support having an average pore size of 5 to 500 nm, said porous support being filled with a polymer which is cross-linked,
wherein the polymer is selected from polyvinylamines or polyallylamines having a weight average molecular weight (Mw) of 2,000 to 500,000 Da and a hydrolysis degree of the formamide groups of at least 66%,
with the proviso that a polyvinylamine having a weight average molecular weight (Mw) of 27,200 Da and a hydrolysis degree of the formamide groups of 70% and a polyvinylamine having a weight average molecular weight (Mw) of 50,000 Da and a hydrolysis degree of the formamide groups of 95% are excluded.

2. The composite material according to claim 1, wherein the porous support is a particulate material with an average particle size of 1 μm and 500 μm.

3. The composite material according to claim 1, wherein the porous support material is porous silica gel.

4. The composite material according to claim 1, wherein the polyvinylamine is a linear or branched homopolymer of vinylamine or a copolymer of vinylamine and vinylformamide.

5. The composite material according to claim 1, wherein the concentration of cross-linked polymer is at least 3% w/w based on the total weight of the dry composite material.

6. The composite material according to claim 1, wherein the hydrolysis degree of the polyvinylamine or polyallylamine is 68% to 99%.

7. A method for producing the composite material according to claim 1 comprising the steps of:
a) soaking a porous support having an average pore size of 5 to 500 nm in a solution or a dispersion containing a polymer, a cross-linker, and a solvent; and
b) cross-linking the polymer with the cross-linker at a temperature below 250° C.,
wherein the polymer is selected from polyvinylamines or polyallylamines having a weight average molecular weight (Mw) of 2,000 to 500,000 Da and a hydrolysis degree of the formamide groups of at least 66%,
with the proviso that a polyvinylamine having a weight average molecular weight (Mw) of 27,200 Da and a hydrolysis degree of the formamide groups of 70% and a polyvinylamine having a weight average molecular weight (Mw) of 50,000 Da and a hydrolysis degree of the formamide groups of 95% are excluded.

8. The method according to claim 7, wherein the solvent is selected from water, alcohols, ethers and ketones, or mixtures thereof.

9. The method according to claim 7, wherein the cross-linker is selected from propanediol diglycidylether, butanediol diglycidylether, hexanediol diglycidylether, glutaric dialdehyde and succinic dialdehyde.

10. Use of the composite material of claim 1 for purifying a target protein in a feedstock.

11. The use according to claim 10, wherein the target protein is a monoclonal antibody.

12. A method for purifying a target protein in a feedstock, said method comprising the steps of:
i) contacting the feedstock with a composite material according to claim 1 for a sufficient time;
ii) separating the composite material from the purified feedstock;
iii) optionally, isolating the target protein from the feedstock; and
iv) optionally; washing the composite material with a solvent and collecting the obtained solution for further processing.

13. The method according to claim 12; wherein the target protein is a monoclonal antibody.

14. The method according to claim 12, wherein the contact time is at least 1 min.

15. The method according to claim 12, wherein the feedstock comprises host cell proteins (HCPs) and DNA.

16. The composite material according to claim 2, wherein the porous support material is porous silica gel.

17. The composite material according to claim 2, wherein the polyvinylamine is a linear or branched homopolymer of vinylamine or a copolymer of vinylamine and vinylformamide.

18. The composite material according to claim 3, wherein the polyvinylamine is a linear or branched homopolymer of vinylamine or a copolymer of vinylamine and vinylformamide.

19. The composite material according to claim 2, wherein the concentration of cross-linked polymer is at least 3% w/w based on the total weight of the dry composite material.

20. The composite material according to claim 3, wherein the concentration of cross-linked polymer is at least 3% w/w based on the total weight of the dry composite material.
